(19) United States
(12) Patent Application Publication
(10) Pub. No.: US 2002/0143322 A1
(43) Pub. Date: Oct. 3, 2002

(54) Apparatus and method for shrinking collagen

(76) Inventor: Ali Z. Haghighi, Sunnyvale, CA (US)

Correspondence Address: K. Alison de Runtz, c/o SKJERVEN MORRILL MacPHERSON LLP, 28th Floor, Three Embarcadero Center, San Francisco, CA, 94111, US

(21) Appl. No.: 09/770,098

(22) Filed: Jan. 23, 2001

(51) Int. Cl. A61B 18/18; A61N 5/06

(52) U.S. Cl. 606/5; 607/91; 607/88; 606/27

(57) ABSTRACT

The present invention provides a device, a system, and associated methods for advantageously treating bodily tissues containing collagen, such as the cornea. The device is a topical device that, when placed over the target tissue, defines a space between an inner surface of the device and the tissue. The radiation-transparent device allows radiation to pass therethrough to the tissue during the radiation treatment. The device is believed to reduce heat loss, such as evaporative heat loss, from the tissue, thereby improving the tissue-heating efficiency of the radiation treatment. Typical radiation treatment parameters may thus be adjusted to provide a more gentle, yet highly efficient treatment using the inventive device. The gentle treatment reduces the likelihood of thermally traumatizing the treated tissue. While the invention has particular application in the area of corneal treatment, it may be used to prepare other tissues or substrates.

### FIELD OF THE INVENTION

The invention relates generally to a device that is usefully placed over a surface of bodily tissue during irradiation of the tissue to facilitate the irradiation process or the achievement of a desired outcome of the irradiation process. More particularly, the invention relates to a device that allows radiation to pass to the tissue surface while reducing heat loss at the tissue surface. The device is usefully employed in the treatment of tissue containing collagen, where the device facilitates the shrinkage of collagen within the tissue. The invention also relates to a system employing such a device and a method of using such a device.

### BACKGROUND OF THE INVENTION

Various techniques for irradiating or thermally treating bodily tissues have been in use for some time. Particular techniques have been directed to the treatment of bodily tissue that contains collagen to change the character of the collagen within the tissue. See, for example, Sand, U.S. Pat. Nos. 4,976,709; 5,137,530; 5,304,169; and 5,484,432 (hereinafter, the “Sand Patents”).

Collagen is known to shrink when heated to a shrinkage temperature of generally from about 50° C. to about 100° C. depending on the type of collagen. For example, Type IV collagen within the cornea is known to shrink when heated to from about 60° C. or about 65° C. to about 75° C. or about 80° C. Thus, in order to shrink collagen within tissue that is typically at body temperature, or well below the shrinkage temperature, sufficient energy must be applied to the tissue to bring it to the shrinkage temperature. Many of the previous techniques used to shrink collagen have used energy that is more aggressive than that needed to shrink the collagen without traumatizing or ablating tissues adjacent the targeted collagen or the targeted collagen itself. Many prior techniques have thus resulted in undesirable tissue trauma.

Undesirable tissue trauma has been a particular concern in the treatment of collagen within a cornea, a treatment that is often used to modify a refractive condition of the cornea. In many refractive modification procedures, the energy used, typically some form of laser energy, is too potent to shrink collagen tissue without ultimately causing thermal trauma to the untargeted tissue, such as the outermost epithelial layer of the cornea, or to the stroma itself. These tissues generally react to excessive heating by developing a haze or cloudiness in the cornea, which may result in vision complications such as glare and/or the appearance of halos. The appearance of corneal haze or opacity would be a significant problem in the treatment of the refractive condition of myopia, as the energy is typically applied to corneal locations within or fairly close to the central optical zone to obtain central corneal flattening. Further, vision complications, such as glare or the appearance of halos, are often associated with treatments performed within the radial area defined by a dilated pupil.

There is therefore a need for a less aggressive collagen modification procedure that reduces or eliminates undesirable tissue trauma. There is a particular need for such a procedure for the correction of refractive conditions of the cornea, such as a photothermal keratoplasty or LTK procedure, wherein a defined pattern of electromagnetic radiation is delivered to an external surface of the cornea in a controlled manner for the purpose of reshaping the cornea.

There are many specific treatment procedures which involve directing a highly controlled beam of electromagnetic radiation to an eye. For example, one specific surgical procedure involves using a radiation beam to ablate and thus cut portions of the corneal tissue. A specific application of this surgical procedure is in the performance of a radial keratotomy procedure, in which radial cuts are made in the cornea using a laser as opposed to a surgical knife. In another specific treatment procedure, an outside surface of the cornea is removed by an excimer laser in order to reshape the cornea. Despite the existence of the aforementioned specific procedures, alternative “keratoplasty” procedures are currently receiving a great deal of attention because of their ability to correct for myopia (nearsightedness), hyperopia (farsightedness), and/or astigmatism.

In a particular keratoplasty procedure, which avoids cutting the cornea, at least one beam of electromagnetic radiation within the infrared portion of the spectrum is directed at the eye to shrink collagen tissue within the cornea in order to cause corrective changes in corneal curvature. This technique, often termed “photothermal keratoplasty”, is the subject of the aforementioned Sand Patents and of U.S. Pat. No. 5,779,696 to Berry et al. (hereinafter, the “Berry et al. Patent”). The aforementioned Sand Patents and the Berry et al. Patent are expressly incorporated herein in their entireties by this reference.

The collagen-shrinkage methods and apparatus of Sand and Berry et al. are disclosed as being applicable for modification of collagen tissue throughout the body. When the tissue is corneal collagen tissue and the radiation source is a laser, such methods are typically referred to as “laser thermokeratoplasty” or “laser thermal keratoplasty” (LTK). These LTK techniques promise to provide permanent changes to the optical characteristics of the human cornea with a higher degree of safety and patient comfort than that provided by techniques that involve physically cutting and removing portions of the cornea.

One way to deliver a desired electromagnetic radiation pattern to the cornea is by projection from a short distance removed from the cornea. One instrument for doing so is described in the Published International Patent Cooperation Treaty Application WO 94/03134 (hereinafter, the “PCT Publication”), which PCT Publication is expressly incorporated herein in its entirety by this reference. This instrument allows an ophthalmologist, or other attending physician or practitioner, to select and deliver a specific pattern and amount of electromagnetic radiation to each patient in accordance with the condition to be corrected. It is desirable for such an instrument to perform efficient corrective photothermal keratoplasty procedures on a large number of patients with a high degree of accuracy, effectiveness, safety and convenience.

The aforementioned Third Co-Pending Application discloses apparatus and methods for applying a flow of a conditioning or drying medium to an external surface of an eye of a patient, to dry the eye in preparation for ophthalmological observation and/or treatment. Such apparatus and methods are particularly useful in preparing a patient's eye for vision-corrective ophthalmological treatments, such as photothermal keratoplasty or LTK. The aforementioned First Co-Pending Application discloses apparatus and methods for advantageously exposing an eye of a patient to a controlled pattern of radiation, while the Second Co-Pending Application discloses coordinated or automated apparatus and methods for so exposing the eye, to provide convenience and to promote efficiency for an attending physician or other provider of the treatment. The aforementioned Fourth Co-Pending Application discloses compositions and methods useful to stabilize a condition of collagenous tissue that results from its modification. The aforementioned Fifth Co-Pending Application discloses optical devices, systems, and methods useful to determine a condition of collagenous tissue, and particularly useful for developing a process for modifying such tissue. The aforementioned Sixth Co-Pending Application discloses coordinated apparatus and methods for advantageously exposing an eye to radiation, particularly corneal and/or scleral portions thereof, which are particularly useful in the treatment of presbyopia.

There remains a need for a relatively gentle tissue modification procedure, particularly such a procedure for the modification of collagen tissue within the cornea.

### SUMMARY OF THE INVENTION

The present invention is directed to a device and a system, and associated methods, useful in the radiation treatment of bodily tissue containing collagen, such as collagenous tissue of the cornea. The device is a topical device that is placed over the tissue undergoing treatment, defining a space between an inner surface of the device and the underlying tissue. The topical device is transparent to the treatment radiation, allowing the treatment radiation to pass through the device to interact with the tissue.

It is believed that when the topical device is placed over the tissue and radiation passes through the device to the tissue, the topical device reduces heat loss from the tissue surface, most particularly, heat loss associated with evaporation. As heat loss is reduced, the efficiency of the heating of the tissue by irradiation is greater than that associated with treating uncovered tissue. Because the topical device effectively holds heat within the tissue, the treatment parameters previously associated with radiation treatment of tissue can be made less aggressive to obtain the desired outcome. This means that the desired outcome can be obtained with a greater margin of safety, such that the likelihood of thermally traumatizing the tissue, particularly the surface tissue, is greatly reduced. The device is therefore particularly advantageous in the radiation treatment of corneal tissue to reduce or eliminate a refractive condition of myopia, as thermal trauma previously associated with such treatment of myopia has led to significant vision complications.

The present invention provides the topical device just described, as well as a system that includes the topical device and a source of radiation. In the inventive system, the radiation source may be any of a variety of sources effective for a particular application, such as a laser. While the invention is most often described in relation to a particular application, namely, the treatment of a cornea, it can be used in the preparation and/or treatment of a variety of substrates, such as non-corneal or non-ophthalmic tissue.

Additional objects, advantages and features of the present invention will become apparent from the description of preferred embodiments, set forth below, which should be taken in conjunction with the accompanying drawings, a brief description of which follows.

### BRIEF DESCRIPTION OF THE DRAWINGS

The drawings include FIGS. 1-6, not all of which are drawn to scale or to the same scale. These drawings are briefly described below.

FIG. 1 is a diagram of a human eye, shown in vertical cross-section along a central axis of the eye, as viewed from the side.

FIG. 2 is a perspective view of an ophthalmic treatment system for producing controlled patterns of treatment radiation, as disclosed in the aforementioned First Co-Pending Application, wherein the view is from a side that faces an attending physician.

FIG. 3 is a schematic, side view of a topical device, according to an embodiment of the present invention, shown in relation to a cross-sectional side view of an anterior portion of a subject's eye.

FIG. 4 is a schematic, side view of a topical device, according to another embodiment of the present invention, shown in relation to a cross-sectional side view of an anterior portion of a subject's eye.

FIG. 5 is a histographical depiction of the results of an Experiment A described herein.

FIG. 6 is a histographical depiction of the results of an Experiment B and an Experiment C described herein.

### DESCRIPTION OF PREFERRED EMBODIMENTS

The invention is now described with reference to the above-described Figures. Reference symbols are used in the Figures to indicate certain aspects or features shown therein, with reference symbols common to more than one Figure indicating like aspects or features shown therein. It should be noted that reference symbols used herein are intended to be internally consistent, such that whether or not they happen to coincide with those used in applications that have been incorporated herein by reference, their meaning will be apparent to those of ordinary skill in the art. While the invention is now described for the most part in relation to the treatment of ocular tissue, and more particularly, corneal tissue, it will be understood that the invention has application to all types of collagenous bodily tissue.

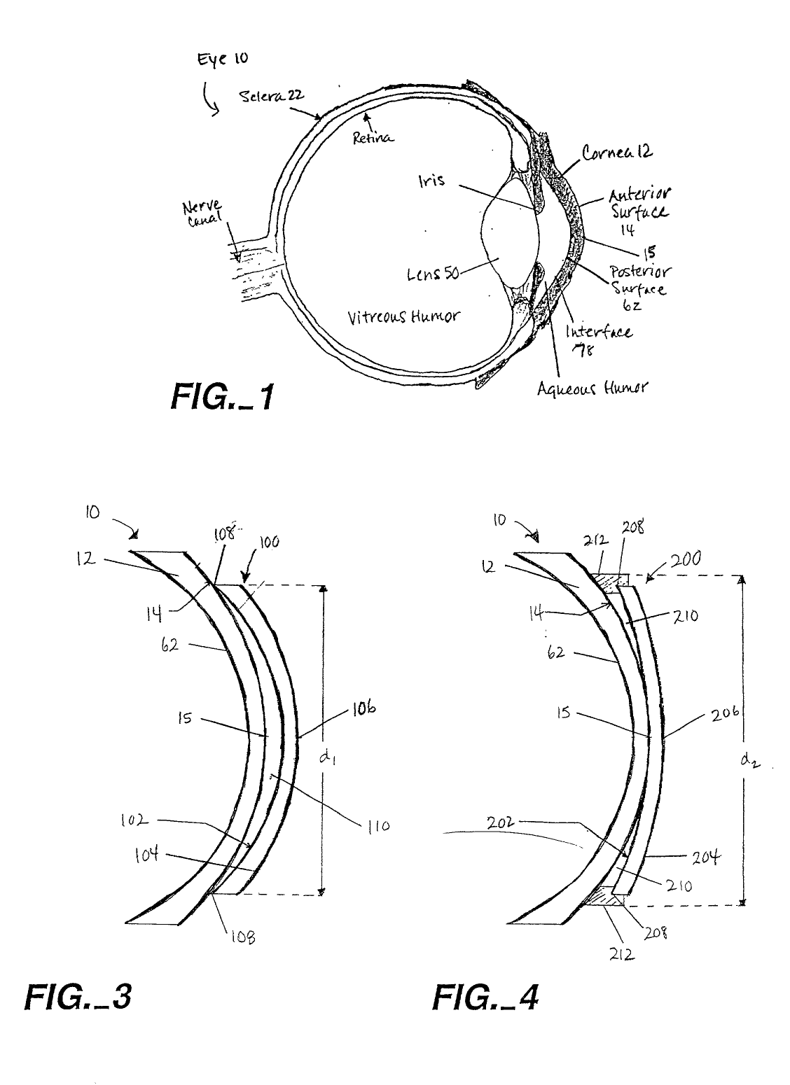

As shown in FIG. 1, the human eye 10 is a roughly spherical structure having a transparent cornea 12 at its forward central portion. At the periphery of the cornea is an opaque sclera 22. The cornea is composed of various layers, as described in U.S. Pat. No. 5,137,530 to Sand. The total thickness of the cornea at its center is about 0.55 millimeters. The outermost or anterior corneal layer 14 is the epithelium (including its underlying basement membrane), which is typically about 50 microns thick and accounts for about 10 percent of the total corneal thickness. Below this epithelial layer lies Bowman's membrane, which is typically about 10 to about 13 microns thick and is non-regenerative. Beneath Bowman's Membrane lies the corneal stroma, which is typically about 90 percent of the total thickness of the cornea and is composed of clear sheets of collagenous material. The corneal stroma is backed by Descemet's Membrane, which is typically about 5 to about 10 microns thick. Finally, the innermost or posterior corneal layer 62 is the endothelium, which layer is typically about 4 to about 5 microns thick and is composed of a single layer of non-reproducing, flattened cells. The internal lens 50 of the eye is posterior to the cornea.

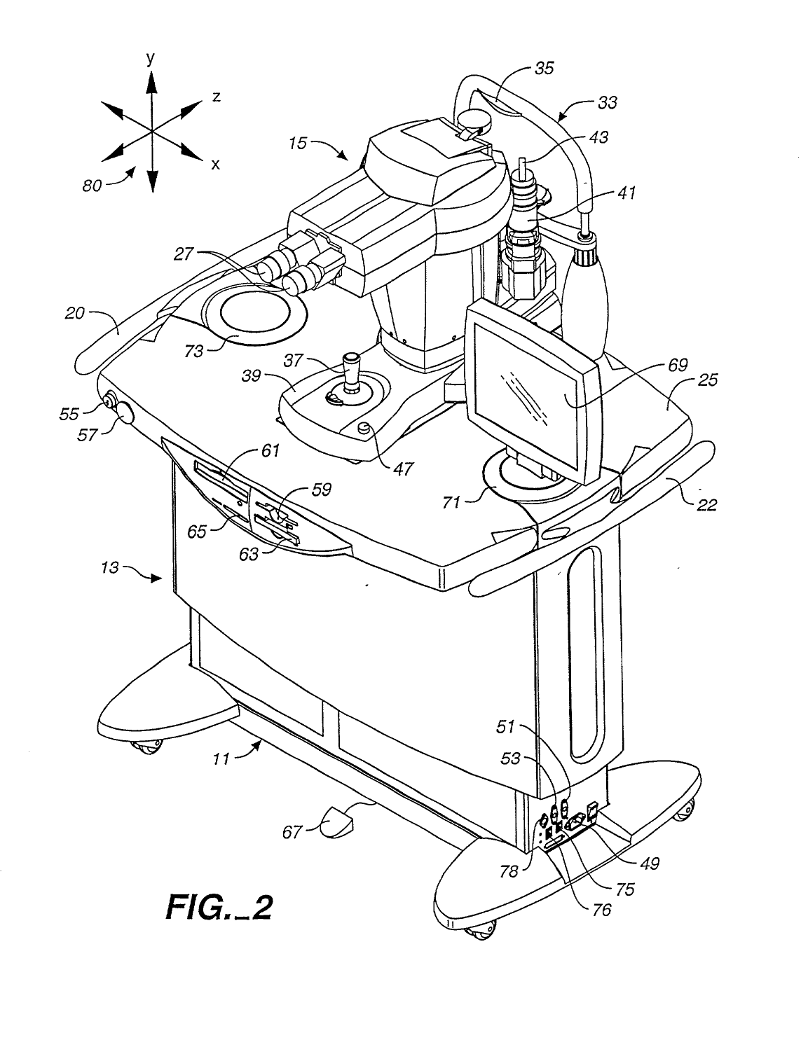

While the geometry of the cornea is complex, it can be described generally as having surfaces which are approximately concentric and spherical. Typically, the epithelial surface 14 of the cornea has a radius of curvature of about 8 millimeters. This radius of curvature is smaller than the average radius of curvature of the sclera 22, such that the cornea has a bulged appearance with respect to the sclera. The diameter of the cornea at its greatest chord is typically about 11 millimeters. While various portions of the eye, including the cornea, have been schematically illustrated in FIGS. 3 and 4, such illustrations should be taken in conjunction with the cross-sectional diagram of the human eye and various of its components, as shown in FIG. 1.

The various aspects of the present invention, those summarized above and others, are illustrated herein to be implemented in a system that corrects vision by photothermal keratoplasty. The system is easily configured to precisely generate a desired pattern of electromagnetic radiation to correct a vision deficiency, such as far-sightedness, of a particular patient. The specific type or amount of vision correction required by the particular patient determines the specific configuration for that patient. Many aspects of the present invention are also applicable to other techniques of eye vision correction, wherein certain parameters are different, such as the treatment radiation wavelengths, patterns, exposure times, and the like. Further, many aspects of the present invention are applicable to the generation of radiation patterns for other uses than correcting vision. Additionally, many aspects of the present invention are applicable to operation of a wide variety of medical treatment or diagnosis systems.

The illustrative instrument is now generally described in relation to FIG. 2. By way of convenience, the system is described herein with reference to terms which correspond to a representation 80 of a three-dimensional Cartesian coordinate system, including an x-axis, a y-axis, and a z-axis. Right, left, lateral, horizontal, or like movement is in a direction substantially parallel to the x-axis; up, down, elevational, vertical, or like movement is in a direction substantially parallel to the y-axis; and fore, aft, proximity-adjusting, or like movement is in a direction substantially parallel to the z-axis.

This instrument is specifically designed for use in an office of an ophthalmologist, other physician or medical service provider, where reliability and ease of use are important since technical assistance is not on site or very close to the office. A base 11 is provided with casters for ease of movement of the system within the office. A table assembly 13 is carried by the base in a manner to be adjustable up and down with respect to the base by a motor (not shown) within the base. This allows vertical adjustment of an optical radiation delivery instrument 15 to suit a physician 17 that is performing the procedure and a particular patient 19 who is having his or her vision corrected. This adjustment, along with a usually independent adjustability of physician and patient chairs 21 and 23, permits comfortable positioning of both the physician and patient with respect to the instrument 15. Handles 20 and 22 on opposite sides of a top 25 of the table of the assembly 13 make it easy to move the system by rolling on its casters.

The radiation delivery instrument 15 is carried on the top 25. During the procedure, the physician looks through binoculars 27 on one side of the instrument 15 and a treatment optical radiation pattern exits the other side of the instrument through an opening 29. This radiation is directed through a few inches of air to a patient eye 31 being treated. Only one eye is treated at a time in one procedure. In order to hold the treated eye in a fixed position with respect to the table assembly 13, a headrest assembly 33 is attached to the table top 25. The headrest assembly 33 is described in more detail in a Published International Patent Cooperation Treaty Application WO 00/13571 (hereinafter, the “Herekar et al. PCT Publication”), which Herekar et al. PCT Publication is expressly incorporated herein in its entirety by this reference.

Briefly, in one operational embodiment, the patient's head is placed in contact with the assembly 33 in preparation for or during treatment. The head is optionally urged against the assembly 33 such as by being strapped against it. A transducer 35 is built into a top of the headrest assembly 33 in a position to be contacted by the forehead of the patient. This transducer provides an electrical signal with a magnitude related to a degree of contact between the patient's forehead and the assembly 33. By way of example, the degree of contact, and thus, the electrical signal, may be related to an amount of pressure or force applied to the assembly 33 when the patient's forehead contacts the assembly. The resulting electrical signal is used to confirm an appropriate level of contact, or to indicate an inappropriate level of contact, between the patient's forehead and the headrest assembly. Thus, this electrical signal is usefully fed into an electronic control portion of the system that, for example, may provide a desired safety response.

Once the patient's head is placed against the headrest assembly 33, the radiation pattern from the opening 29 is manually aligned with the eye 31 by movement of the optical instrument with respect to the table top 25. The physician so moves the instrument by manipulating a joystick type of handle control 37 on a base 39 of the instrument. The handle 37 operates a mechanism (not shown) positioned under the base 39 of the instrument 15 that, in response to movement of the handle 37 to the left or right by the physician 17, moves the projected radiation pattern between the patient's right and left eyes and horizontally adjusts the pattern on the selected eye 31 being treated. Movement of the handle 37 forward and backward by the physician 17 moves the instrument 15 toward and away from the patient, respectively, to control the focus of the radiation pattern on the eye 31 being treated. Vertical motion of the instrument 15 with respect to the table top 25 is not provided in this example, but could also be provided. Rather than moving the instrument 15 up and down with respect to the table top 25, the vertical position of the patient eye 31 being treated is controlled by a mechanical adjustment of the headrest assembly 33.

Included as part of the optical instrument 15 is an illuminator 41 that directs light through a top prism 43 to the patient eye being treated from a side of the eye. This illuminates the eye so that the physician 17 may have a clear view of it through the binoculars 27 when carrying out the treatment procedure. The illuminator 41 is rotatable by hand with respect to the instrument 15 about an axis (not shown). The attending physician may easily adjust the angle of the eye illumination, while looking through the binoculars, in order to obtain a good view of the eye being treated. The illuminator 41 will generally be rotated to one side or the other, depending upon whether the right or left eye of the patient is being treated. Since the prism 43 directs light from about the same height as the treatment radiation output 29, it is rotated out of the way when treatment radiation is directed against the patient eye 31. The intensity of light from the illuminator 41 is adjusted by the physician through rotation of a knob 47 on the base 39 of the instrument. Alternatively, an illuminator may be housed within an optical instrument (not shown) which is equipped with appropriate illuminator controls, such as a modified optical instrument 15.

The base 11 includes a number of electrical receptacles for connection to power and communications systems. Included are a receptacle 49 for a power cord, a receptacle 51 for a telephone line and a receptacle 53 for a local area network (LAN). Several controls and devices are provided on the physician's side of the table assembly 13. These include a key-operated power switch 55 and an emergency button 57 that turns off the treatment radiation source. A floppy-disk drive 59 is also positioned on a side of the table facing the physician. A compact-disk (CD) drive 61, a high-capacity, removable-disk drive 63 and a slot of a card interface 65 for removably receiving an electronic card are also provided. Many of the radiation sources used in the system and a controlling computer are installed in the base unit 11. A foot switch 67 is provided for the physician to use to start treatment after the system is adjusted for a particular patient eye.

A primary input/output device to the system's controlling computer system is a touch-sensitive screen 69. It can be mounted to the table top 25 on either the right (as shown) or left side of the physician, by attachment to respective receptacles 71 and 73. Thus, the attending physician may select whichever side is the most convenient. A usual computer keyboard may also be connected to the internal computer system through a receptacle 75 in the base unit 13 but will unlikely be used by the physician to perform treatments since the touch screen 69 is usually preferred. A tray (not shown) can be added to extend the table top 25 to support a keyboard. A keyboard will be useful when a significant amount of data are input or retrieved through the treatment system, rather than though another computer connected in a LAN with the treatment system. Standard computer-peripheral receptacles 76 and 78 are also provided for connection to an external printer and monitor, respectively. Additional details of the system shown in FIG. 2 are given in the First Co-Pending Application referenced above.

The system for irradiating tissue, as just described, may be a coordinated or automated system as described in the Second Co-Pending Application referenced above. More particularly, and preferably, the system may be coordinated such that computer software may be used to implement a variety of treatments. An example and a preferred example of computer software for implementing treatments are provided in source code in the microfiche appendices that are part of the Second and Sixth Co-Pending Applications, respectively. These source codes are subject to copyright protection by Sunrise Technologies International, Inc., assignee of the present application. The copyright owner has no objection to the facsimile reproduction by anyone of the above-mentioned appendices, as they appear in the Patent and Trademark Office patent file or records, but otherwise reserves all copyright rights whatsoever.

The above-described system may be used to correct undesirable or abnormal refractions of a patient's eye by delivering energy in a pattern of spots to the patient's cornea such that the curvature of the cornea is modified. The system can be used for a variety of refractive conditions, including astigmatism, and is particularly useful in the treatment of hyperopia wherein the curvature of the cornea is steepened to increase the refractive power of the cornea. Whatever the initial condition of the cornea, the treatment provider considers that condition, chooses an appropriate treatment plan, and treats the patient accordingly.

The wavelength of radiation used for the treatment, its magnitude and the duration of the exposure, are selected to be adequately absorbed by the corneal or other exposed tissue to raise its temperature at the exposed spots to a level sufficient so that the tissue changes in the manner desired. When used to reshape the surface of the cornea or other tissue, the preferred technique is to control these parameters to cause tissue below the surface to change in a manner that reshapes the surface, without ablating the exposed tissue. The radiation wavelength is usually selected from the infrared or near infrared portions of the spectrum. However, the instruments and techniques described herein are also applicable to other processes that require exposure to radiation patterns with different parameters.

Once the treatment plan is finalized, the user or treatment provider may prepare to treat the selected patient eye. Typically, the user will precondition or dry the eye. Preferably, the eye is dried to reduce or eliminate a tear film that may otherwise compromise or interfere with the corneal-modification treatment. The upper and lower eye-lids may be held out of the corneal area, for example, using a speculum, to facilitate eye-drying. The eye may be dried using a flow of drying medium, such as warm (about 45° C.), dry air (about 15% relative humidity), for a period of from about 7 seconds to about one minute, and preferably, from about 15 seconds to about 30 seconds, as described in the Third Co-Pending Application. Preferably, the temperature and relative humidity are well controlled, as may be accomplished using the conditioning system described in the above-mentioned application. This is the preferred conditioning method, as it minimizes eye-preparation time and provides substantially uniform eye-drying. Alternately, the eye may be dried naturally by the presence of ambient air in the vicinity of the eye over a period of time, such as three minutes. Natural drying is not preferred given the time involved and the lack of control over the ambient conditions, which may affect drying uniformity, and thus, the treatment outcome, and repeatability from one treatment to other treatments.

Once the eye is in a condition appropriate for treatment, the collagen tissue is exposed at the selected location to radiation sufficient to raise the collagen temperature to a shrinkage temperature of from about 50° C. to about 100° C., or preferably from about 50° C. or about 60° C. to about 80° C., or more preferably from about 65° C. to about 75° C. when the collagen is Type IV collagen, in the manner disclosed herein above and in the First-Sixth Co-Pending Applications mentioned above. Any suitable radiation source may be used, such as a laser, a source of incoherent radiation, a source of radiofrequency radiation, a source of microwave radiation, a source of ultrasonic radiation, and a source of thermal radiation, such as a tissue-contacting source of thermal radiation, or an electrical source of thermal radiation. The source of heat may be pulsed or intermittent, or continuous. Preferably, the radiation source is a laser, such as a pulsed laser emitting radiation of a wavelength of between about 1.4 or about 1.8 to about 2.55 microns, or a pulsed Ho:YAG laser emitting radiation of a wavelength of about 2.12 microns. Alternately, the radiation source may be one emitting radiation of a wavelength that corresponds to a tissue absorption coefficient of from about 10 to about 100 cm⁻¹, particularly when the tissue is corneal tissue. Upon radiation exposure, the collagen tissue reaching the shrinkage temperature will shrink. Collagen fibrils have been reported to shrink to up to about ⅓ of their pre-treatment length when heated to shrinkage temperature.

Preferably, the tissue undergoing modification is treated according to a treatment plan designed to produce the desired outcome, such as any of the treatment plans described in the First-Sixth Co-Pending Applications. It will be understood that these First-Sixth Co-Pending Applications simply provide examples of possible treatment plans, as there are numerous possible treatments which effectively account for variations in the treatment parameters. By way of example, treatment parameters subject to variation include the selection of a single treatment region or a number of treatment regions, the size of a treatment region, the shape of a treatment region, the pattern used to treatment a treatment region, such as the number or size of spots in the pattern, the intensity of irradiation, the duration of irradiation, the selection of a single pulse of radiation or a number of pulses of radiation from a pulsed source, the selection of a continuous source, as well as various other treatment parameters. Further, treatment plans may be designed to treat a variety of tissue, such as corneal tissue of the eye and/or scleral tissue of the eye, as disclosed in the Sixth Co-Pending Application, or any other bodily tissue containing collagen, such as connective tissue or musculoskeletal tissue found throughout the body, as disclosed in the Sand Patents. Treatment plans may also be designed to treat a variety of conditions, such as refractive conditions of myopia, hyperopia, presbyopia, and/or astigmatism; ocular conditions, such as accommodation for near vision; cosmetic conditions, such as wrinkles or undesirable cosmetic appearance; musculoskeletal conditions, such as injury to musculoskeletal connective tissue; connective tissue conditions, such as lack of elasticity of connective tissue; otological conditions, such as a looseness or lack of elasticity in the tympanic membrane, as described in U.S. Pat. No. 5,591,157 of Hennings et al.; a variety of conditions of different tissues, as described in the Sand Patents, such as an undesirable condition of a heart valve; and a wide variety of other conditions.

Examples of systems and methods for treating tissue containing collagen have been described. According to the present invention, a device for placement over a surface of such tissue is advantageously employed in connection with such systems and methods. An example of one such device is now described in relation to FIG. 3.

FIG. 3 schematically illustrates a front portion of the eye 10, including the cornea 12 and its anterior surface 14 and posterior surface 62. For a typical human eye, the radius of curvature of the anterior surface 14 is about 7.8 millimeters to about 8.0 millimeters. Naturally, this radiation of curvature varies from subject to subject, such as from about 7.6 millimeters to about 8.5 millimeters. As schematically shown in FIG. 3, a device 100 is placed in useful proximity to an anterior or external surface 14 of the cornea 12 of a subject's eye 10. The device 100 may be placed in relation to the anterior corneal surface in any manner sufficient to pass light from a radiation source, such as a radiation source of the radiation delivery instrument 15 of FIG. 2, to the cornea. By way of example, the device 100 may be held in relation to the corneal surface by any sufficient means, such as by a holder (not shown), whether manually held, if practical, or fixed to a support structure.

According to a preferred embodiment, the device 100 is placed on the anterior surface of the cornea, much in the way that a vision-corrective contact lens is placed on the anterior surface of the cornea. Thus, preferably, the device 100 has a concave posterior surface 102 that facilitates its placement or retention on the corneal surface 14. The shape of the anterior surface 104 of the device is less important, although preferably it is of a shape that is comfortable for the subject and does not interfere with the efficient passage of radiation therethrough. The device 100 may be referred to as a lens, for convenience, though it differs from the conventional vision-corrective lens in that it does not need to conform to the outer surface of the cornea in the way that a vision-corrective lens typically does, as further described below.

That is, as shown in FIG. 3, the posterior surface 102 of the device 100 has a radius of curvature that is less than the radius of curvature of the anterior surface 14 of the cornea. By way of example, the radius of curvature of the posterior surface 102 may be less than the typical human radius of curvature of the anterior surface of about 7.8 to about 8.0 millimeters, such as about 6.5 millimeters to about 7.7 millimeters, or about 7.3 millimeters. Further, the device 100 has a diameter d₁ that is less than or equal to the diameter of the anterior surface 14 of the cornea over which it is placed. By way of example, the diameter d₁ may be about 9 to about 11 millimeters. When the device 100 is placed over the corneal surface, as shown, portions of the device, such as the end portions 108 furthest from the center 106 of the device, will contact anterior surface 14 of the cornea. When so placed, the device defines a space 110 between its posterior surface 102 and the anterior surface 14 of the cornea. This space 110 has greater dimensions, such as volume or depth, than those associated with a conventional contact lens placed on the surface of the cornea. For example, the space may be greater than about 0.1 mm in depth, such as from about 0.15 mm or about 0.2 mm or more, or from about 0.15 mm or about 0.2 mm to about 0.8 mm. Further by way of example, the space may have a volume of from about 0.002 cm³ to about 0.05 cm³. While this space may be partially filled with tear fluid or film, or some other topical medium present on the anterior surface 14 of the cornea, the space is of a dimension sufficient to be at least partially, if not substantially, filled with a gaseous medium, such as ambient gas, and most typically, air.

According to the present invention, the device 100 is placed over the corneal surface before the cornea is irradiated and remains in place during irradiation by any of the irradiation methods previously described. Preferably, prior to this placement of the device 100, the corneal surface is dry, wiped dry, or pre-treated to dryness, as previously described, to remove at least some of the natural tear fluid or other topical fluid from the corneal surface. As described above, the device 100 is of a construction sufficient to pass light from a selected radiation source to the cornea. By way of example, the device may be made of a radiation-transparent material such as glass, sapphire, quartz, a crystalline material, a plastic or polymeric material, or any combination thereof.

When radiation is passed through the device 100 to the tissue surface, the temperature of the cornea increases. Topical fluids, such as tear fluid, absorb the radiation before the underlying cornea does, such that these fluids reach a higher temperature than is desirable at the point of irradiation. Further, this absorption of heat by the topical fluids reduces the heat available to the stromal tissue of the cornea. For these reasons, it is desirable to dry the cornea in advance of treatment, as described above. During irradiation, heat may be lost or dissipated from the stroma by conduction, convection, radiation and/or evaporation. For the time periods considered here, such as about 1 second, conduction, convection and radiation are inefficient means of cooling the irradiated stroma. Thus, in the irradiation process described above, the primary heat loss mechanism is believed to be heat loss associated with evaporation. By way of explanation, it is believed that evaporation takes place after radiation heating, such that water vapor from the tissue surface enters the gas-filled space of the topical device 100. As the gas-filled space approaches or achieves saturation, further evaporation is inhibited or eliminated. The device 100 thus effectively reduces heat loss from the tissue surface during an irradiation procedure of one or more applications of radiation, particularly heat loss associated with evaporation.

A particular advantage of the device 100 just described is that it holds heat within the tissue. This means that the treatment parameters previously used to heat the tissue sufficiently to obtain a desired outcome, can be reduced, or made less aggressive, such that substantially the same outcome is obtained with a greater safety margin. The likelihood of thermally traumatizing tissue, particularly the surface tissue, is thus greatly reduced. As used herein, thermal trauma refers to one or more of the following conditions: ablation of the tissue surface, necrosis of the tissue surface or the cells thereof, and hazing of the tissue surface or stroma.

The device 100 is particularly useful when used in connection with a modification procedure designed to treat myopia. In the treatment of myopia, radiation is typically applied to the central part of the cornea, such as at radial distances of about 1.5 millimeters to about 2.0 millimeters from the center 15 of the cornea, or diameters across the cornea of about 3 millimeters to about 4 millimeters. This treatment region is close to, or coincidental with, the central visual portion of the cornea. When the treatment is too aggressive, the epithelial or surface tissue in the treatment region may be burned or ablated or may become cloudy. Such trauma may lead to vision complications, such as glare or the appearance of halos. Thus, a more gentle treatment procedure is particularly desirable for the treatment of myopia.

The device 100 allows for a more gentle treatment for myopia. As shown in FIG. 3, the space 110 defined by the device encompasses the central region of the cornea. A less aggressive radiation regime may be used to heat the central region of the cornea, as the central space 110 serves to prevent heat loss from the corneal surface, thereby enhancing the efficiency of corneal heating to achieve the desired outcome. According to a particular treatment regime, laser energy is applied via device 100, as described above, in an annular pattern of spots at radial distances of about 1.5 millimeters to about 2.0 millimeters from the center 15 of the cornea. A pattern of eight spots may be used, such as a pattern of eight spots generated by the system described herein and in the First and Second Co-Pending Applications.

According to this treatment regime, the radiation may be of a wavelength of from about 1.4 to about 2.55 microns, such as the radiation generated from a Ho:YAG laser of a wavelength of about 2.12 microns. Alternately, the radiation may be of a wavelength corresponding to a tissue absorption coefficient from about 10 cm⁻¹ to about 100 cm⁻¹. In any event, the radiation is sufficient to heat the collagen within the targeted tissue to its shrinkage temperature of from about 50° C. to about 100° C., or preferably from about 50° C. or about 60° C. to about 80° C., or more preferably from about 65° C. to about 75° C. The radiation is applied in pulses, such as from about 5 to about 50 pulses, and preferably, a series of about 10 to about 20 pulses depending on the desired correction. The radiation provides a total energy of from about 50 mJ to about 250 mJ, and preferably, about 100 mJ per eight-spot pattern over a duration of about 10 to about 20 pulses. The energy density is from about 2 to about 11 J/cm², preferably from about 4 to about 6 J/cm², such as 4.25 J/cm². This energy density may also be expressed as being from about 6 to about 32 mJ per spot of irradiation on the surface of the cornea. The power is from about 250 mW to about 1.25 W per eight spots. The overall period of treatment or irradiation, including the pulses of irradiation and the intervals therebetween, is from about 1 to about 60 seconds, preferably from about 2 to about 10 seconds, and more preferably from about 2 to about 4 seconds. The frequency of the pulses is about 5 pulses per second, each pulse having a duration of about 125 microseconds and the interval between consecutive pulses having a duration of about 200 milliseconds.

According to the present invention, the device 100 effects the shrinkage of collagen within the targeted tissue such that an amount of radiation energy effective to shrink the collagen with the device is less than that effective to shrink the collagen without the device, which is typically from about 200 to about 250 mJ per eight spots. The device 100 thus allows for a less aggressive treatment, such that significant trauma or ablation of the surface of the targeted tissue is substantially avoided. Further, the device 100 effects collagen shrinkage such that a number of radiation pulses or a period of irradiation effective to shrink the collagen with the device is greater than that effective to shrink the collagen without the device. By way of example, about 13 pulses of relatively low energy (about 100 mJ per 8 spots) may be used when the device is employed, while about 5-10 pulses of comparatively high energy are typically needed when the device is not employed. This means that a slower, more gentle treatment can be used when the device is employed. The slower, more gentle treatment is not effective when the device is not used.

An example of another device for placement over a surface of tissue, that is advantageously employed in connection with the treatment systems and methods described herein, is now described in relation to FIG. 4. FIG. 4 schematically illustrates a front portion of the eye over which a device 200 has been placed, much in the manner described above in relation to the device 100 of FIG. 3. Preferably, the device 200 is placed on the anterior surface of the cornea, much in the way that a vision-corrective contact lens is placed on the anterior surface of the cornea. Thus, preferably, the device 200 has a concave posterior surface 202 that facilitates its placement on the corneal surface 14. The shape of the anterior surface 204 of the device is less important, although preferably it is of a shape that is comfortable for the subject and does not interfere with radiation transmission therethrough. As illustrated, the device 200 has side portions 212, which may be integral to or attached to the device, that facilitate both its placement and its retention on the corneal surface. Such side portions 212 may also be employed with the device 100 of FIG. 3. These side portions 212 may be composed of a heat-insulative material, to further reduce heat loss from the tissue surface, although it is believed that such insulation means do not significantly contribute to heat loss reduction.

The device 200 may be referred to as a lens, for convenience, though it differs from the conventional vision-corrective lens in that it does not need to conform to the outer surface of the cornea in the way that a vision-corrective lens typically does, as further described below. That is, as shown in FIG. 4, the posterior surface 202 of the device 200 has a radius of curvature that is greater than the radius of curvature of the anterior surface 14 of the cornea. By way of example, the radius of curvature of the posterior surface 202 may be greater than the typical human radius of curvature of the anterior surface of about 7.8 millimeters to about 8.0 millimeters, such as about 9 millimeters to about 11 millimeters. Further, the device 200 has a diameter d₂ that is less than or equal to the diameter of the anterior surface 14 of the cornea over which it is placed. By way of example, the diameter d₂ may be about 9 millimeters to about 11 millimeters. When the device 200 is placed over the corneal surface, as shown, a portion of the device, such as a central portion in a vicinity of the center 206 of the device, will contact anterior surface 14 of the cornea. When so placed, the device defines a space 210 between its posterior surface 202 and the anterior surface 14 of the cornea. This space 210 has greater dimensions, such as volume or depth, than those associated with a conventional contact lens placed on the surface of the cornea. For example, the space 210 may have dimensions that are the same as, or similar to, those described above in relation to the space 110 of device 100. Further by way of example, the volume may be from about 0.01 cm³ to about 0.05 cm³. While this space may be partially filled with tear fluid or film, or some other topical medium present on the anterior surface 14 of the cornea, the space is of a dimension sufficient to be at least partially, if not substantially, filled with a gaseous medium, such as ambient gas, and most typically, air.

According to the present invention, the device 200 is placed over the corneal surface before the cornea is irradiated and remains in place during irradiation by any of the irradiation methods previously described. The device 200 reduces heat loss during irradiation in much the same manner as that previously described in relation to the device 100 of FIG. 3.

The device 200 is particularly useful when used in connection with a modification procedure designed to treat hyperopia. In the treatment of hyperopia, radiation is typically applied to a region beyond the central part of the cornea, such as at radial distances of about 3.0 millimeters to about 4.0 millimeters from the center 15 of the cornea, or diameters of about 6 millimeters to about 8 millimeters. This treatment region is beyond the central visual portion of the cornea. When the treatment is too aggressive, the epithelial or surface tissue in the treatment region may be burned or may become cloudy. This trauma does not typically lead to vision complications because this surface tissue lies beyond the central visual portion of the cornea. Nonetheless, this surface trauma is generally undesirable. Thus, a more gentle treatment procedure is desirable for the treatment of hyperopia.

The device 200 allows for a more gentle treatment for hyperopia. As shown in FIG. 4, the space 210 defined by the device corresponds to an outer region beyond the central region of the cornea. A less aggressive radiation regime may be used to heat this outer region of the cornea, as the device holds heat within the tissue surface, which heat can be used to assist in the heating of the tissue. By way of example, a modification of the treatment regime described above in relation to device 100 of FIG. 3 may be used with the device 200 of FIG. 4 to treat hyperopia in a gentle and effective manner. According to this modified regime, the laser energy described above is applied via device 200, as described above, in an annular pattern of spots at radial distances of about 3.0 millimeters to about 3.5 millimeters from the center 15 of the cornea. A pattern of eight spots may be used, such as a pattern of eight spots generated by the system described herein and in the First and Second Co-Pending Applications. In all other respects, the treatment regime is substantially the same as that described in relation to the treatment regime employed when using the device 100 of FIG. 3. With the topical device 200, the treatment parameters previously used to heat the tissue sufficiently to obtain a desired outcome, can be toned down or made less aggressive, such that substantially the same outcome is obtained with a greater safety margin. The likelihood of thermally traumatizing tissue, particularly the surface tissue, is thus greatly reduced.

While two particular embodiments of the topical device of the present invention have just been described, other variations are contemplated as being within the scope of the invention. By way of example, the radius of curvature of the device 100 or 200 may substantially correspond to that of the outer surface of the tissue being treated, or the device may be variably or non-uniformly curved or even uncurved, where devices such as the side devices 212 are employed at various locations to create the space 110 or 210, or another space or other spaces, between the device and the tissue surface, as suitable for the particular treatment contemplated.

Further, while two particular embodiments of the topical device have been described in connection with the treatment of the cornea, the device may be suitably configured for placement over other bodily tissue undergoing a modification procedure where a reduction of heat loss from the tissue is desirable. For example, the device may be used for the treatment of a scleral portion of the eye, as disclosed in the Sixth Co-Pending Application. In such an application, the topical device is configured such that the device covers the scleral portion of the eye and defines a space between the scleral tissue and the device when placed over the eye, the topical device is placed over the eye, suitable radiation is passed to the scleral tissue through the device, and heat loss from the irradiated scleral tissue is reduced to effect the scleral treatment. For such a scleral treatment, the tissue may be raised to a shrinkage temperature of from about 60° C. to about 100° C., as there are no known vision complications associated with treating the scleral tissue to temperatures higher than the high-end threshold of about 80° C. associated with corneal tissue. In another example, the topical device is suitably configured to cover bodily tissue containing collagen, such as the epidermis of skin tissue slated for modification, while leaving a space between the epidermis and the device, when placed over the epidermis, and a suitable radiation procedure is carried out as described above to raise the collagen tissue to a shrinkage temperature appropriate for the type of collagen being treated.

The topical device described herein may be incorporated into a system for treating bodily tissue containing collagen, such as the system of FIG. 2. In such a system, the topical device is placed over the tissue to be treated and the tissue is oriented with respect to the radiation source. The tissue is then irradiated through the topical device with suitable radiation from the radiation source to shrink collagen within the tissue. While the system of FIG. 2 is designed for the treatment of ocular tissue, it will be understood that any system having a source of radiation suitable for a particular treatment application, and optionally, an orientation system suitable for orienting the particular tissue to be treated with the radiation source, may by used with the topical device described herein, in a system and method suitable for treating bodily tissue, as described herein.

### EXPERIMENTS

Experiments relating to various aspects of the present invention are now described.

### Experiment A

This experiment was undertaken to compare changes in the curvature of porcine corneas that occur when the corneas are exposed to radiation either with or without the topical device of the present invention. The corneal epithelium was removed from each of the porcine corneas undergoing testing to promote absorption of a preparatory solution in the corneal stroma. This preparatory solution of 7.5% dextran in a saline solution was used to make the porcine corneas more like human corneas. Each of the porcine corneas was soaked in this preparatory solution for at least 30 minutes. Topographical maps of these corneas were then taken using the EyeSys Videokeratoscope commercially available from EyeSys Laboratories of Houston, Tex. Each of the corneas was then rinsed with saline to remove the preparatory solution and manually wiped dry. Six corneas grouped in two sets (Sets 1A and 3A) of three porcine corneas were then covered with a sapphire topical device 100 (FIG. 3) of the present invention, while another six corneas grouped in two other sets (Sets 2A and 4A) of three porcine corneas remained uncovered, in preparation for the irradiation treatment further described below.

The LTK treatment procedure was carried out using the SUN 1000™ Corneal Shaping System commercially available from Sunrise Technologies International, Inc. of Fremont, Calif. In these procedures, the radiation was focused on the corneal surface. For one of the sets of covered corneas (Set 1A) and for one of the sets of uncovered corneas (Set 2A), the irradiation pattern consisted of an 8-spot circular pattern centered on the cornea and having a diameter of about 3 millimeters. These corneas were irradiated according to this pattern, using twenty pulses of radiation and an energy of 100 mJ per 8 spots. For the remaining set of covered corneas (Set 3A) and for the remaining set of uncovered corneas (Set 4A), the irradiation pattern consisted of a 16-spot circular pattern centered on the cornea and having a diameter of about 3 millimeters. These corneas were irradiated once using an 8-spot irradiation pattern as described above for Sets 1A and 2A, and subsequently irradiated using another 8-spot irradiation pattern also as described for Sets 1A and 2A, with the exception that the second 8-spot pattern was rotated 20° with respect to the first 8-spot pattern to produce a 16-spot circular pattern of spots approximately equally spaced along the circumference of the 16-spot circle. Following irradiation, the corneas were once again topographically mapped using the Eyesys Videokeratoscope.

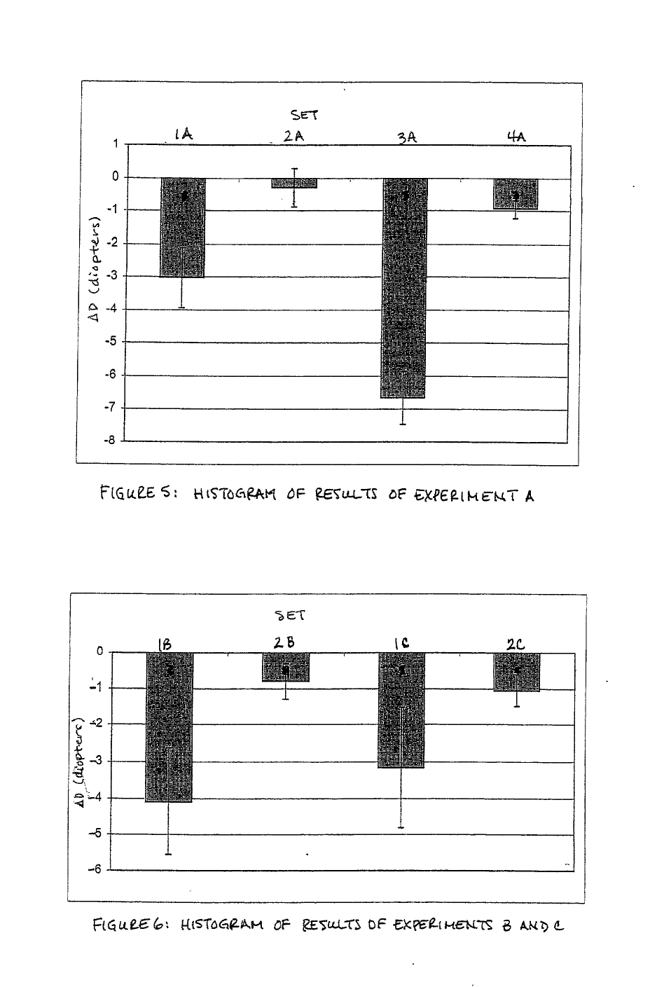

The post-irradiation data that were collected are now described. As described above, for each cornea, a topographical map was obtained before irradiation and another topographical map was obtained after irradiation, using the Eyesys Videokeratoscope. The Eyesys Videokeratoscope may be used to compare the curvature of the cornea at any point on the pre- and post-irradiation topographical maps. The Eyesys Videokeratoscope was so used in this experiment to determine the change between the pre- and post-irradiation topographical maps in diopters at various sample points on the corneal surface, such as at the center of the cornea and/or at the flattest portion of the cornea, and ultimately, to obtain the lowest value so determined for this change in diopters (ΔD). The results of this experiment are tabulated in Table 1, for each of the three eyes in each of the sets (Sets 1A-4A), along with the average and standard deviation for each of the sets. The average and standard deviation for each of these sets are also shown histographically in FIG. 5. The histogram of FIG. 5 clearly demonstrates that the corneas in the experimental sets (Sets 1A and 3A), in which a topical device of the present invention was used, showed significantly greater diopter changes than those in the experimental sets (Sets 2A and 4A), in which the cornea was uncovered.

TABLE 1
Results of Experiment A

| Set | 1A | 2A | 3A | 4A |
|---|---|---|---|---|
| ΔD (diopters) | −4.29 | −0.76 | −7.71 | −1.03 |
| ΔD (diopters) | −2.22 | −0.67 | −5.73 | −1.22 |
| ΔD (diopters) | −2.55 | 0.52 | −6.56 | −0.58 |
| Average ΔD (diopters) | −3.020 | −0.303 | −6.667 | −0.943 |
| Standard Deviation (diopters) | 0.908 | 0.583 | 0.812 | 0.268 |

According to Experiment A, greater diopter changes are associated with corneal irradiation via the topical device of the present invention than with irradiation of an uncovered cornea. Further experiments (Experiments B and C, described below) were then conducted to determine what might be responsible for these greater changes associated with use of the topical device. For example, when the device is used, any evaporation that occurs might lead to condensation on the inside of the device, which condensation might scatter light passing through the device toward the cornea. Experiment B was undertaken to compare the diopter change associated with corneal irradiation via the device and the diopter change associated with irradiation of a bare cornea using defocused light to approximate the effect of light which is scattered before reaching the cornea. Further by way of example, when the device is used, any evaporation that occurs might lead to an accumulation of heat associated with evaporation between the device and the corneal surface. Experiment C was undertaken to compare the diopter change associated with intermittent corneal irradiation via the device, where the device remains on the cornea throughout the irradiation process, and the diopter change associated with intermittent corneal irradiation via the device, where the device is removed from the cornea during the intervals in which irradiation is interrupted. Experiments B and C are now described in greater detail.

### Experiment B

This experiment was undertaken to determine the effect of possible light scattering on the treatment outcome of porcine corneas treated according to the present invention. The corneas tested in this experiment were prepared for irradiation and topographically mapped in the same manner described above in relation to Experiment A. Each of four corneas in one set (Set 1B) of the porcine corneas was then covered with a sapphire topical device of the present invention, while each of two corneas in another set (Set 2B) of the porcine corneas remained uncovered, in preparation for irradiation treatment. The corneas then received the same 8-spot irradiation treatment that was used for Sets 1A and 2A in Experiment A, with the exception that for the uncovered corneas in Set 2B, the radiation beam was slightly defocused. The corneas were topographically mapped following this treatment.

The Eyesys Videokeratoscope was used in this experiment in the same manner it was used in Experiment A to determine the change between the pre- and post-irradiation topographical maps in diopters at various sample points on the corneal surface, such as at the center of the cornea and/or at the flattest portion of the cornea, and ultimately, to obtain the lowest value so determined for this change in diopters (ΔD). The results of this experiment are tabulated in Table 2, for each of the four eyes in Set 1B and for each of the two eyes in Set 2B, along with the average and standard deviation for each of the sets. The average and standard deviation for each of these sets are also shown histographically in FIG. 6. The histogram of FIG. 6 clearly demonstrates that the corneas in the experimental Set 1B, in which a topical device of the present invention was used and the corneas were irradiated with focused radiation, showed significantly greater diopter changes than those in the experimental Set 2B, in which the cornea was uncovered and irradiated using defocused light. It is believed that light scatter is not a significant mechanism responsible for the large diopter changes associated with corneal irradiation via the topical device of the present invention.

### Experiment C

This experiment was undertaken to determine the possible heat accumulation effect on the treatment outcome of porcine corneas treated according to the present invention. The corneas tested in this experiment were prepared for irradiation and topographically mapped prior to irradiation in the same manner described above in relation to Experiment A. Each of four corneas in one set (Set 1C) and five corneas in another set (Set 2C) of the porcine corneas was then covered with a sapphire topical device of the present invention in preparation for irradiation treatment. The corneas then received the same 8-spot irradiation treatment that was used for Sets 1A and 2A in Experiment A, with the exception that for both of Sets 1C and 2C, an interrupted sequence of 25 pulses of radiation was delivered to the corneas. More particularly, each of the corneas in Set 1C received a series of 5 pulses of radiation separated by 10-second intervals in which no radiation was delivered, until the 25 pulses of radiation were delivered. In Set 2C, each of the corneas received a series of 5 pulses of radiation separated by intervals during which the topical device was removed (allowing any heat to dissipate), wiped clean (removing any condensate), and replaced, until the 25 pulses of radiation were delivered. The corneas were topographically mapped following this treatment.

The Eyesys Videokeratoscope was used in this experiment in the same manner it was used in Experiment A to determine the change between the pre- and post-irradiation topographical maps in diopters at various sample points on the corneal surface, such as at the center of the cornea and/or at the flattest portion of the cornea, and ultimately, to obtain the lowest value so determined for this change in diopters (ΔD). The results of this experiment are tabulated in Table 2, for each of the four eyes in Set 1C and for each of the five eyes in Set 2C, along with the average and standard deviation for each of the sets. The average and standard deviation for each of these sets are also shown histographically in FIG. 6. The histogram of FIG. 6 clearly demonstrates that the corneas in the experimental Set 1C, in which a topical device of the present invention was used and remained in place during the intervals in which irradiation was interrupted, showed significantly greater diopter changes than those in the experimental Set 2C, in which a topical device of the present invention was used, but was removed and wiped clean during the intervals in which irradiation was interrupted. It is believed that heat accumulation is a significant mechanism responsible for the large diopter changes associated with corneal irradiation via the topical device of the present invention.

TABLE 2
Results of Experiment B and Experiment C

| Set | 1B | 2B | 1C | 2C |
|---|---|---|---|---|
| ΔD (diopters) | −5.42 | −1.30 | −2.88 | −1.73 |
| ΔD (diopters) | −1.72 | −0.31 | −5.28 | −1.37 |
| ΔD (diopters) | −5.14 | | −0.73 | −0.49 |
| ΔD (diopters) | −4.11 | | −3.79 | −0.76 |
| ΔD (diopters) | | | | −0.90 |
| Average ΔD (diopters) | −4.098 | −0.805 | −3.17 | −1.05 |
| Standard Deviation (diopters) | 1.457 | 0.495 | 1.649 | 0.444 |

The invention is described herein with particular reference to the treatment of the cornea, as that is a particularly sensitive application that fairly teats the capability of the invention. While so described, the invention may be used in a variety of ophthalmic applications where radiation treatment of the cornea or eye is desired. For example, the topical device may be used to treat ophthalmic tissue associated with a corneal transplantation or may be used in pre- or post-surgical treatments of ophthalmic tissue, including touch-up or re-treatment. The invention may also be used to treat other non-corneal or non-ophthalmic bodily tissue by simply positioning the topical device or treatment system appropriately in relation to the target tissue and proceeding with the treatment of that tissue. The invention thus has many useful applications including a great variety of treatments for correcting an undesirable condition of selected tissue by modifying a shape, structure, or appearance of the tissue being treated. By way of example, the invention may be used to treat a tissue wound, a surgical site, tissue having a cosmetically undesirable condition, such as skin having wrinkles, and the like.

Various aspects and features of the present invention have been explained or described in relation to beliefs or theories, although it will be understood that the invention is not bound to any particular belief or theory. Further, although the various aspects and features of the present invention have been described with respect to the preferred embodiments thereof, it will be understood that the invention is entitled to protection within the full scope of the appended claims.

It is claimed:

1. A device for placement over a surface of tissue containing collagen, said device having an exterior surface and an interior surface; defining space between the interior surface and the tissue surface when said device is placed over the tissue surface, said space being at least partially filled with a gaseous medium; and being of a construction sufficient to pass radiation to the tissue surface when so placed, said radiation sufficient to effect a shrinkage of collagen within the tissue; wherein said device is of a construction sufficient such that heat loss from the tissue surface when said radiation is passed to the tissue surface via said device is less than heat loss from the tissue surface when said radiation is passed to the tissue surface without said device.

2. The device of claim 1, wherein the tissue surface is the anterior surface of the cornea.

3. The device of claim 1, wherein said device effects the shrinkage of collagen within the tissue such that an amount of radiation energy effective to shrink the collagen via said device is less than that effective to shrink the collagen without said device.

4. The device of claim 1, wherein an amount of radiation energy effective to shrink the collagen via said device is from about 2 to a bout 11 J/cm².

5. The device of claim 1, wherein the radiation is in a form of at least one spot and an amount of radiation energy effective to shrink the collagen via said device is from about 6 to about 32 mJ per spot.

6. The device of claim 1, wherein said device effects the shrinkage of collagen within the tissue such that a period of irradiation effective to shrink the collagen without thermally traumatizing the tissue surface via said device is greater than that effective to shrink the collagen without thermally traumatizing the tissue surface without said device.

7. The device of claim 6, wherein said period of irradiation effective to shrink the collagen without thermally traumatizing the tissue surface via said device is from about 1 to about 60 seconds.

8. The device of claim 1, wherein said device effects the shrinkage of collagen within the tissue such that a number of radiation pulses effective to shrink the collagen without thermally traumatizing the tissue surface via said device is greater than that effective to shrink the collagen without thermally traumatizing the tissue surface without said device.

9. The device of claim 8, wherein said number of pulses effective to shrink the collagen without thermally traumatizing the tissue surface via said device is from about 5 to about 50.

10. The device of claim 1 or 2, wherein said device effects the shrinkage of collagen within the tissue while substantially avoiding ablation of the tissue surface.

11. The device of claim 1 or 2, wherein said device effects the shrinkage of collagen within the tissue while substantially avoiding necrosis of the tissue surface.

12. The device of claim 1 or 2, wherein said device effects the shrinkage of collagen within the tissue to obtain a post-treatment state, and reduces regression of the post-treatment state toward a pre-treatment state.

13. The device of claim 2, wherein said device effects the shrinkage of collagen within the tissue while substantially avoiding hazing of the tissue surface.

14. The device of claim 2, wherein said device effects the shrinkage of collagen within the tissue while substantially avoiding hazing of stromal tissue of the cornea.

15. The device of claim 1, wherein said device is composed of a radiation-transparent material.

16. The device of claim 2, wherein said device is effective to enhance the shrinkage of collagen within the cornea to modify a shape of the cornea.

17. The device of claim 2, wherein said device is effective to effect the shrinkage of collagen within the cornea to alter a refractive condition.

18. The device of claim 17, wherein the refractive condition is selected from a group consisting of myopia, hyperopia, astigmatism, presbyopia, and any combination thereof.

19. The device of claim 1, wherein the interior surface has a radius of curvature that is less than that of the tissue surface.

20. The device of claim 1, wherein the interior surface has a radius of curvature that is greater than that of the tissue surface.

21. The device of claim 20, wherein a substantially central portion of the interior surface contacts the tissue surface, such that the space is defined by portions of the interior surface outside of the substantially central portion.

22. The device of claim 1, wherein the interior surface has a radius of curvature that is about equal to that of the tissue surface.

23. The device of claim 20 or 22, further comprising a structure sufficient to locate the interior surface over the tissue surface and to define the space therebetween.

24. The device of claim 1, further comprising a heat insulator at the periphery of the device.

25. The device of claim 1, wherein the space has a volume of from about 0.002 cm³ to about 0.05 cm³.

26. The device of claim 1, wherein the space has a volume of from about 0.01 cm³ to about 0.05 cm³.

27. The device of claim 1, wherein the radiation is of a wavelength of from about 1.4 to about 2.55 microns.

28. The device of claim 1, wherein the radiation is of a wavelength corresponding to a tissue absorption coefficient of from about 10 cm⁻¹ to about 100 cm^{-1.}

29. The device of claim 1, wherein the radiation is sufficient to heat the collagen within the tissue to a temperature of from about 50° C. to about 80° C.

30. The device of claim 1, wherein a source of radiation is selected from a group consisting of a source of incoherent radiation, a source of radiofrequency radiation, a source of microwave radiation, source of ultrasonic radiation, a tissue-contact source of thermal radiation, an electrical source of thermal radiation, a source of infrared radiation, a laser, and any combination thereof.

31. The device of claim 30, wherein the source is selected from a group consisting of a pulsed source and a continuous source.

32. The device of claim 1, wherein said device reduces heat loss associated with evaporation from the tissue surface.

33. A system for shrinkage collagen within tissue, comprising the device of claim 1 and a source of radiation sufficient to pass the radiation to the tissue surface via the device.

34. A method of shrinking collagen within tissue, comprising placing the device of claim 1 over the tissue surface and passing the radiation to the tissue surface via the device.
